(19) United States
(12) Patent Application Publication
(10) Pub. No.: US 2014/0128671 A1
(43) Pub. Date: May 8, 2014

(54) Trocar System

(71) Applicants: Siegfried Riek, Rottweil (DE); Karl-Heinz Bachmann, Villingendorf (DE); Thomas Gaiselmann, Villingendorf (DE)

(72) Inventors: Siegfried Riek, Rottweil (DE); Karl-Heinz Bachmann, Villingendorf (DE); Thomas Gaiselmann, Villingendorf (DE)

(21) Appl. No.: 14/129,881

(22) PCT Filed: Jun. 4, 2012
(86) PCT No.: PCT/EP2012/002353

§ 371 Date: Dec. 27, 2013

(30) Foreign Application Priority Data
Jun. 30, 2011 (DE) 10 2011 107 612.7

(51) Int. Cl. A61B 1/00; A61B 1/018; A61B 1/04; A61B 17/30; A61B 1/06; A61B 17/29; A61B 17/3201; A61B 17/34; A61B 1/012

(52) U.S. Cl. CPC A61B 1/00154; A61B 17/3423; A61B 1/018; A61B 1/04; A61B 1/0125; A61B 1/06; A61B 17/3474; A61B 17/3478; A61B 17/29; A61B 17/3201; A61B 17/30
USPC 600/104

(57) ABSTRACT

A trocar system, having a trocar, an optical channel extending coaxially in the trocar for receiving an optical unit, and a hollow transparent distal tip of the trocar, which can be observed from the interior by means of the optical unit, wherein at least one working channel is constructed in the wall of the trocar surrounding the optical channel, which channel extends continuously and axially parallel from the proximal end of the trocar into the distal tip and opens into an outlet opening in the region of the tip.

The invention relates to a trocar system according to the preamble of claim 1.

Trocar systems that are intended for use in minimally invasive surgical applications typically consist of a trocar that is used to create an opening in a body cavity (for example, the abdomen) and a trocar sleeve that is placed and remains inside said opening constituting an access point to the inside of the body cavity for the surgical procedure. The trocar includes a distal tip for penetrating the body tissues, for example the abdominal wall, and serves to create an opening. The tip of the trocar can be configured as pointed, cutting or dull. A pointed tip, for example, has the shape of a three-edged pyramid. Cutting tips include a blade for a tissue incision that is subsequently dilated by a cone-shaped tip. Dull tips are distally rounded, which means that very high penetration pressures must be applied to them if they are used for opening up tissue layers. Correspondingly, dull tips are essentially only used to dilate a previously created lesion.

These trocars, particularly pointed and cutting trocars, are associated with risks; upon penetrating the abdominal wall, they may cause injury to internal organs that can adhere to the peritoneum due to internal adhesions, such as, for example, the bowel and/or blood vessels in the abdominal wall or retroperitoneum. To reduce this risk, so-called optical trocars are in use, for example, as disclosed in U.S. Pat. No. 5,685,820 A. The distal tip on these optical trocars is configured as a hollow, transparent cone that can be observed from the inside through an optical unit, which is taken up inside an optical channel extending coaxially inside the trocar. With the transparent tip, the optical trocar gives access to a three-dimensional view of the tissue layers of the abdominal wall through which the trocar passes. This affords the surgeon with a sensed idea for the motion, speed and position of the trocar tip during penetration. In particular, it is possible to detect any adhesions that may be present between the bowel and the peritoneum at the insertion point prior to penetrating the peritoneum. Nevertheless, the high penetration pressures needed for passing through the fascia and the peritoneum still remain problematic. Although, conceivably, it is possible to reduce the necessary penetration pressures by the use of cutting blades that are disposed on the trocar tip, the use of a blade poses new injury risks for the bowel during the penetration step. To reduce the penetration pressure, and as a compromise between reducing pressure and injury risk, the known optical trocar is used with or without scraping runners, wherein, however, even in this case, the penetration pressures are still relatively high, and permanently rotating trocar motions are required. Moreover, there results the so-called tenting effect, whereby the trocar presses the tissue layers that require high penetration pressures in a tent-like fashion into the abdomen, possibly advancing them into close proximity of the retroperitoneum. When these layers are opened, they give way to the penetration pressure suddenly, and the tip penetrates the abdomen all of a sudden, possibly making it difficult for the surgeon to control the sudden trocar motion in an effort to avoid injuring internal organs or large vessels in the retroperitoneum with the tip of the trocar. To avoid this problem, many surgeons work with a mini-laparotomy. This procedure envisions the placement of a skin incision according to the classical technique through the abdominal wall, the abdomen is opened and a trocar sleeve then inserted into the opened peritoneum.

Sealing a pneumoperitoneum is problematic herein, because the opening in the abdominal wall, which is created in this way, is larger than the opening that would have been created with a trocar-driven perforation. The open incision of the abdominal wall is in contradiction, however, to the stated goal of a minimally invasive surgical technique.

Therefore, it is the object of the present invention to provide a trocar that utilizes all of the advantages of the optical trocar without requiring, however, high pressures for the tissue penetration.

According to the invention, this object is achieved by a trocar system that has the characterizing features as set forth in claim 1.

Advantageous embodiments of the invention are indicated in the dependent claims.

The essential inventive idea provides for using the trocar not only as a passive tool that is manually guided by an axial force and, if necessary, a rotational movement through the tissue layers. Rather, the trocar according to the invention can be associated with various active surgical instruments that are inserted through at least one working channel, which is configured inside the trocar, and can be extended at the distal tip of the trocar. This allows the surgeon to conduct surgical work directly at the distal tip of the trocar, without the need of having to create a further access point in addition to the trocar. Taking advantage of the optical unit and the transparent distal tip of the trocar, the surgeon has visual contact while executing the surgical procedures by means of the instruments that are extended through the working channels. A large number of different instruments is available in miniaturized design configuration and that are suitable for traversing the working channels. A corresponding multitude of different surgeries is thus made possible with the trocar system according to the invention.

For the insertion of the trocar through the different tissue layers, particularly the abdominal wall, it is possible to provide a miniaturized pair of scissors or a blade, which are extended through the distal trocar tip, to thereby separate or cut into the respective tissue layer in front of the distal tip. Particularly the resilient tissue layers, for example, of the fascia and the peritoneum can be opened in this manner while maintaining visual contact; a small incision is placed, followed by the subsequent penetration of the tip of the trocar into this incision without applying any major pressure, particularly avoiding the tenting effect, then dilating the incision and penetrating the tissue layer. Similarly to the open laparotomy technique, in this way, it is possible to prepare the visualized tissue layers that rest against the distal tip of the trocar in order to allow for an almost pressureless, and thereby risk-free, penetration of the tissue layers. The semi-transparency of the peritoneum allows for detecting adhesions prior to opening the tissue, while the optical trocar is advanced penetrating the tissue. This is not possible with an open laparotomy.

Furthermore, it is possible to guide pairs of tweezers or forceps through the working channels to hold the tissue at the distal trocar tip, which can be advantageous particularly when an incision is placed by means of a pair of scissors or a blade inserted through a further working channel.

Moreover, with the first perforation it is, furthermore, advantageously possible to insert a miniaturized Veress needle through the working channel. Using this Veress needle, it is possible to perforate the peritoneum under visual contact in order to then insufflate the abdominal cavity by means of the Veress needle.

Moreover, it is also possible to guide clamps or coagulation instruments through the working channels, and extending the same through the distal tip. Using these instruments, it is possible to clamp and/or coagulate such vessels.

Moreover, it is also possible to extend miniaturized morcellators through the distal tip.

Moreover, it is also possible to insert a miniaturized digital camera through a working channel, for example, for the purpose of documenting the surgery from a different perspective. It is, furthermore, possible to insert a fiber-optic light guide for additional illumination means through the working channel.

Therefore, the trocar system according to the invention thus allows for penetrating, in particular, the abdominal wall, by inserting the trocar, for example, until the distal tip of the trocar reaches the fascia. A clamp is then extended through one working channel that holds the fascia, while a pair of scissors is extended through another working channel that is used to open the fascia. This step is achieved while the surgeon has visual contact through the transparent tip of the trocar. The tip of the trocar is now inserted in the thus created opening in the fascia, and wherein the further dilatation is achieved without tissue trauma and with minimal penetration pressure. When the tip of the trocar reaches the peritoneum, it is determined following shifting of the bowel synchronous to the breath that no adhesions are present, whereafter the peritoneum can be opened correspondingly by the scissors and, if necessary, a clamp. The tip of the trocar is then inserted into the opening in the peritoneum that is obtained in this manner. No remarkable penetration pressure is needed here as well, whereby the tenting effect and any of the related associated risks are avoided. This process is analogous to the usual preparative steps in the context of an open laparotomy. However, in contrast to this known preparative process, no larger incision is necessary than the size cut that is needed for inserting the trocar. Alternately, once the peritoneum has been reached, it is possible to extend a Veress needle through a working channel by which the peritoneum is then penetrated under visual contact to then insufflate the abdomen with carbon dioxide (CO2). As soon as, due to the insufflation, the peritoneum has been separated from the bowel, the Veress needle is retracted, and the tip of the trocar is inserted through the opening that has been created in this manner in order to dilate said opening without causing tissue trauma and with the application of minimal pressure.

In the trocar system according to the invention, it is possible to use the trocar to place a trocar sleeve, which then serves as an access channel for the subsequent minimally invasive surgery. In the same manner, it is possible to conduct a surgery through a single port using the trocar system without placing a trocar sleeve. In this instance, the trocar remains, along with the optical unit and the working channels, the only access point for the subsequent minimally invasive surgery, wherein the surgical instruments are inserted through the working channels.

The instruments that are used in connection with the trocar system according to the invention are essentially miniaturized surgical instruments that are known from the prior art. They include an extendable working element at the distal tip of the trocar, while, on the end that remains proximally outside of the working channel, the proximal actuating elements of the miniature instruments are disposed. The instruments can be configured therein with a rigid or with a flexible shaft. Flexible instruments can be elastically preloaded in such a manner that the distal working elements thereof bend relative to the center axis of the trocar upon exiting from the distal tip of the trocar to allow for executing preparative work directly in front of the transparent tip.

Alternately, it is also possible to dispose a small guide tube, axially displaceable and rotatable, inside the working channel, through which the miniature instrument is traversed. In the distal end region thereof, the guide tube is elastically preloaded to bend. The guide tube is preferably made of a memory alloy with super-elastic properties, for example of nitinol. When the small guide tube is distally pushed out of the working channel, the distal end thereof curves away from the longitudinal axis that is defined by the working channel, wherein the angle of deflection relative to the longitudinal axis increases the farther the distal end of the small guide tube exits from the working channel. By rotating the small guide tube inside the working channel, it is possible to rotate the direction of the deflection around the longitudinal axis. By axially displacing and rotating the small guide tube, it is thus possible to exercise a three-dimensional control over the direction of exit and the positioning of the distal working element of the miniature instrument. Adjusting means, that are provided at the proximal end, allow for the axial and rotational movement of the small guide tube inside the working channel.

To prevent gas from escaping from the insufflated abdominal area, a valve can be envisioned to provide a proximal seal for the working channels, when no instrument is present inside the working channel. A valve of this kind can be formed, in particular, by a sealing lip, which is known from the prior art, that permits an instrument to pass through it and then seals such an inserted instrument along the external circumference thereof.

Furthermore, unused working channels can be sealed off by a mandrin that closes off the distal outlet opening of the working channel to prevent contaminants from entering the working channel.

In terms of manufacturing, the trocar can include a solid wall that encloses the coaxial optical channel, and inside which the working channel are configured as axially parallel, continuous bores. Alternately, it is possible to configure the trocar as having a double wall that forms a hollow annular gap. The working channels are disposed inside this annular gap as axially parallel continuous tubes. If a sufficient amount of space is radially available, the working channels can also be mounted as small tubes on the interior or exterior jacket surface of the wall; and/or, in the case of a plastic trocar, the small tubes can be molded as well. The number of the working channels is determined based on the purpose of use. In the simplest case scenario, only a single working channel is provided. However, preferably, two or three working channels are provided that are disposed at the same mutual angular distances. The distal outlet openings of the working channels can be disposed in the same axial positions in the jacket surface of the distal tip of the trocar. In the same way, it is possible for the working channels to open in distally varying axial positions at the tip of the trocar. The working channels substantially extend in an axially parallel manner inside the trocar, or also in an angular fashion relative to the instrument axis, or helically about the instrument axis. At the proximal end, the working channels can enter in an angular fashion relative to the trocar axis, and/or they can exit in an angular fashion relative to the trocar axis at the distal end. An angular inlet end can, if necessary, facilitate the inserting and handling of the miniature instrument. An angular outlet end can simplify the positioning of the working element of the miniature instrument, particularly if said element is elastically preloaded for bending. The inlet and the outlet openings of the working channel must not necessarily be disposed at the proximal and distal ends of the trocar, respectively; they can also be disposed as axially offset relative to the ends of the trocar.

Breast surgery has been identified as a possible new area of application for the trocar system according to the invention. Tumors or even lymph nodes can be addressed under visual contact, for example by a cosmetically favorable circumareolar incision. At the tumor site, all of the required surgical instruments can then be applied through the working channels. This single-port technique, where the trocar also serves in the role of administering the instruments in the context of the actual surgical procedure, is particularly advantageous from a cosmetic perspective, because only a single incision is required. Moreover, using a working channel, it is possible to execute the carbon dioxide insufflation to create an extended space, such that the preparative surgery can also take place in a tissue cavity that is not already present. The entire surgery therein can be implemented under visual contact through the distal tip of the trocar, such that an exact and complete preparation and removal of the tumor is possible. The use of all of the known surgical instruments and techniques is possible, such as, for example, blades, punches, coagulation instruments, morcellators, optical units, fiber-optic light guides, illumination systems, and the like.

The invention will be explained in further detail below based on the embodiment as illustrated in the drawings. Shows are as follows:

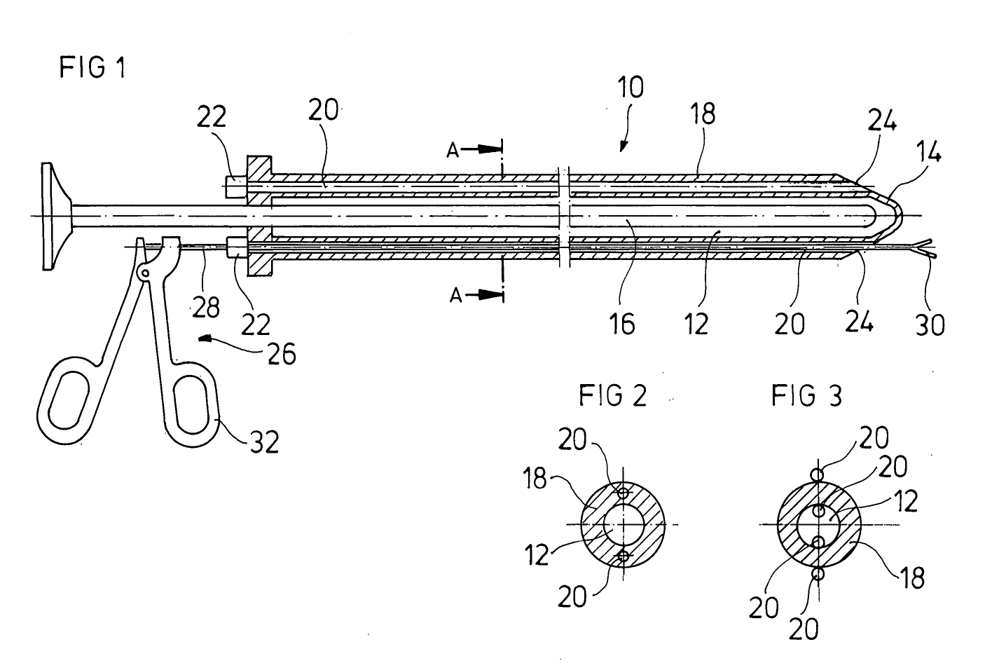

FIG. 1 is a representation of an axial section of a trocar system;

FIG. 2 is a representation of a section along the line A-A in FIG. 1; and

FIG. 3 is a representation of a section corresponding to FIG. 2, by way of a variation of the embodiment.

The trocar system includes a trocar 10. The trocar 10 has the shape of a rigid, oblong, cylindrical tube manufactured of metal or plastic. The internal lumen of the trocar 10 forms a coaxially extending optical channel (12) from the proximal end to the distal end. The distal tip 14 of the trocar 10 is cone-shaped with a rounded, blunt tip. The jacket of the cone can also be convexly arched, if necessary. The cone-shaped tip 14 is hollow on the inside and made of a thin-walled, transparent material, particularly a transparent plastic. An optical unit 16 can be inserted into the optical channel 12 of the trocar, which can be configured, in particular, as a rod-lens optics system or as having a camera chip, and an illumination system can be integrated therein. When an optical unit 16 is inserted, the distal end thereof is disposed approximately in the region of the base area of the cone-shaped tip 14. Due to the optical unit 16, the distal tip 14 can be illuminated and observed from the inside. Thereby, it is possible to observe the tissue that rests against the exterior of the distal tip 14.

Up to this point, the trocar system corresponds to an optical trocar as known from the prior art. Known variations of said optical trocars are also possible and can be used as well according to the invention.

At least one working channel 20 is provided inside the wall 18 of the trocar 10 that encloses the optical channel 12. In the depicted embodiment, two working channels 20 are provided that are diametrically disposed relative to each other. Other embodiments that provide for three or even more working channels 20 are possible as well, which are, in that case, disposed around the optical channel 12 and offset relative to each other, preferably at identical angular distances, respectively.

The working channels 20 extend continuously inside the wall 18 of the trocar 10, from proximal to distal. In the embodiment as depicted in the FIGS. 1 and 2, the working channels 20 are configured as bores inside the solid wall 18. Alternately, the wall 18 can also be formed by two coaxial, sleeve-like tubes having the working channels 20 disposed therein as small, thin tubes inside the circular cylindrical free intermediate space there between. If the inside diameter of the optical channel 12 is greater than the diameter of the optical unit 16 that is to be used, the working channels 20 can also be attached as small tubes to the interior jacket surface of the wall 18. If the outside diameter of the trocar 10 is smaller than the inside diameter of a used trocar sleeve, the working channels 20 can also be attached as small tubes to the outside of the exterior jacket surface of the wall 18. These two possibilities are shown in FIG. 3. The trocar 10 can be manufactured, in particular, of plastic. In this case, it is possible to produce the working channels 20 during the casting step of the plastic; or they can be spray-molded as a tube with the plastic coating material. If the trocar is made of a transparent plastic, it is possible to observe the working channel 20 and any miniature instrument that is inserted into the working channel 20 from the outside.

In a configuration for abdominal surgery, for example, it is possible for the trocar 10 to have an outside diameter of 14 mm, with the optical channel 12 having a diameter of 5 mm, and the working channels 20 having a diameter of 3 mm, respectively.

At the proximal end, the working channels 20 are preferably sealed by a valve 22, which seals the proximal end of the respective working channel 20 in a gas-tight fashion, when the working channel 20 is empty, and which permits the sealed traversal of an instrument there through. A valve 22 of this kind can be configured in a manner as known according to the prior art, for example as a sealing lip, or the like.

The working channels 20 generally take an axially parallel course inside the wall 18 of the trocar 10. At the proximal end, it is possible for the working channels 20 to be outwardly offset, if necessary, relative to the center axis of the trocar 10. In the same way, the working channels 20 can be outwardly offset relative to the axis of the trocar 10 at the distal end thereof. At the distal end, the working channels 20 open through an open outlet opening 24 in the area of the distal tip 14. The outlet openings 24 can be disposed therein in the same axial position with regard to the longitudinal extension of the trocar 10 as shown in FIG. 1. However, it is also possible for the outlet openings 24 of the different working channels 20 to be disposed as axially offset relative to each other, whereby one outlet opening 24 of a working channel 20 opens distally further toward the distal the tip 14 than another outlet opening 24, which can also be disposed, for example, proximally behind the tip 14.

The working channels 20 can accommodate the insertion of miniature instruments 26, respectively, as depicted in FIG. 1 for the bottom working channel 20, for example. The miniature instruments 26 are any type of instrument compliant with the application at hand, as known according to the prior art. The miniature instruments 26 include an oblong shaft 28, which has disposed at the distal end thereon one working element 30, respectively, that can be actuated by means of an actuating element 32 that is disposed at the proximal end of the shaft 28. The drawing depicts, only by way of an example, a miniature pair of scissors, having a working element 30 that is configured as a pair of scissors, while the actuation element 32 is designed as a handle of a pair of scissors.

The miniature instrument 26 is inserted from the proximal end into the working channel 20, wherein it passes through the valve 22 and is sealed by the same along the instrument circumference. The miniature instrument 26 can be advanced inside the working channel 20 until the distal working element 30 is extended through the outlet opening 24 and can be used in the surgical field in front of the distal tip 14. Miniature instruments 26 are known in rigid, flexible and semi-flexible designs. The miniature instruments 26 can be configured, in particular, with an elastically preloaded shaft curvature. This way, the distal working element 30 is deflected from the axial direction of the working channel 20, when said distal working element exits through the outlet opening 24. This allows for targeted positioning of the working element 30 during use.

In an advantageous embodiment, a small guide tube can be inserted in the working channel 20 that can be axially moved and rotated inside the working channel. The small guide tube is made of a memory alloy with so-called super-elastic properties, for example nitinol. The small guide tube is preloaded to curve at least in the distal end area. When the small guide tube is distally advanced inside the working channel 20, such that the distal end of the small guide tube exits from the outlet opening 24, the distal end of the small guide tube curves away from the axial direction of the working channel, wherein the deflection from the axial direction increases the farther the small guide tube exits from the outlet opening. Furthermore, by rotating the small guide tube inside the working channel 20, it is possible to rotate the direction of the distal end of the small guide tube about the axis. When the miniature instrument 26 is then guided through the small guide tube, the direction of the exit of the working element 30 can be controlled in three dimensions by axially displacing and rotating the small guide tube. Adjusting means are provided on the proximal end of the trocar system that facilitate the axial and rotational movements of the small guide tube.

If necessary, it is possible to insert a mandrin into a working channel 20 that is not in use—not shown in the drawing—that will then seal the distal end of the outlet opening 24 in a flush design, whereby any penetration of contaminants into the unused working channel 20 is prevented.

Aside from the trocar 10, the trocar system can also include a trocar sleeve that is pushed onto the trocar 10. Tissue layers, for example, in the abdominal wall are penetrated by means of the trocar 10, and wherein the trocar sleeve, which has been placed on the trocar 10, is inserted into the created body opening. After the trocar 10 has been pulled out, the trocar sleeve remains as an access point to the body cavity.

Alternately, the trocar 10 can also be used as a single port, without the trocar sleeve. After the penetration, the trocar 10 is guided through the created body opening, with the surgery inside the body cavity being conducted under visual contact through the transparent distal tip 14 and using the miniature instruments inserted through the working channels 20.

### LIST OF REFERENCE SIGNS

10 Trocar

12 Optical channel

14 Distal tip

16 Optical unit

18 Wall

20 Working channels

22 Valve

24 Outlet opening

26 Miniature instrument

28 Shaft

30 Working element

32 Actuating element

1. A trocar system, comprising:
a trocar, having an optical channel that extends coaxially inside the trocar for receiving an optical unit, the trocar having a hollow transparent distal tip that can be observed from the interior by the optical unit, wherein a wall of the trocar, which encloses the optical channel, includes a working channel that continuously extends from proximal end to distal end of the trocar and opens into an outlet opening.

2. The trocar system according to claim 1,
wherein the distal tip has the shape of a bluntly rounded cone that is convexly arched in a distal direction.

3. The trocar system according to claim 2,
wherein the outlet opening of the working channel is disposed in the area of the distal tip.

4. The trocar system according to claim 1,
wherein at least two working channels are provided, which are offset relative to each other at an angle of circumference and with outlet openings that are disposed in the same axial positions or positions that are axially offset relative to each other.

5. The trocar system according to claim 1,
wherein the working channel is a bore that extends axially inside the wall of the trocar.

6. The trocar system according to claim 1,
wherein the working channel is configured as a small tube that is disposed in a free intermediate space of the trocar wall, which is configured as a jacketed double wall, on the interior jacket surface of the trocar wall or on the exterior jacket surface of the trocar wall.

7. The trocar system according to claim 1,
wherein the working channel is proximally closed by a valve.

8. The trocar system according to claim 1,
wherein there is a miniature instrument, which can be guided through the working channel in such a manner that a distal working element (30) of the miniature instrument exits at the distal end from outlet opening of the working channel, and in that a proximal actuating element of the miniature instrument is located at the proximal end outside of the working channel.

9. The trocar system according to claim 8,
wherein the miniature instrument is a pair of scissors, a blade, a pair of tweezers, a clamp, a coagulation instrument, a Veress needle, a digital camera, an optical means, a fiber-optic light guide or an illumination system.

10. The trocar system according to claim 8,
wherein a guide tube is disposed, axially displaceable or rotatable, inside the working channel, through which the miniature instrument can be guided, and in that the guide tube is elastically preloaded for curve at least in the distal end region thereof.

11. The trocar system according to claim 10,
wherein the guide tube is made of a memory material having super-elastic properties.

12. The trocar system according to claim 1,
wherein a mandrin can be inserted in the working channel, and in that, in an inserted state, the same closes the outlet opening of the working channel.
